(19) United States
(12) Patent Application Publication
(10) Pub. No.: US 2026/0090916 A1
(43) Pub. Date: Apr. 2, 2026

(54) TREATMENT APPARATUS AND METHOD FOR PROVIDING CONTROL DATA FOR AN OPHTHALMOLOGICAL LASER OF A TREATMENT APPARATUS FOR GENERATING A LASER-INDUCED REFRACTIVE INDEX CHANGE

(71) Applicants: SCHWIND eye-tech-solutions GmbH, Kleinostheim (DE); Clerio Vision, Rochester, NY (US); UNIVERSITY OF ROCHESTER, Rochester, NY (US)

(72) Inventors: Pascal NAUBEREIT, Laufach (DE); Samuel ARBA MOSQUERA, Aschaffenburg (DE); Len ZHELEZNYAK, Rochester, NY (US); Wayne KNOX, Rochester, NY (US); Steve COX, Rochester, NY (US)

(21) Appl. No.: 18/901,587

(22) Filed: Sep. 30, 2024

(51) Int. Cl. A61F 9/008

(52) U.S. Cl. CPC A61F 9/00804

(57) ABSTRACT

The invention relates to a treatment apparatus and a method for providing control data for an ophthalmological laser (12) of a treatment apparatus (10) for generating a laser-induced refractive index change, comprising, as steps, determining (S10) irradiation positions (16) for irradiation by laser pulses for the laser-induced refractive index change, wherein first and second laser pulse lengths (28, 30) are provided for the irradiation of the irradiation positions, wherein the first laser pulse length (28) has a shorter laser pulse length than the second laser pulse length (30); setting (S12) preset irradiation positions (24) to the second laser pulse length (30); and providing (S14) the control data for controlling the ophthalmological laser (12), which includes the adjusted second laser pulse length (30) for the preset irradiation positions (24).

### FIELD

The invention relates to a method for providing control data for an ophthalmological laser of a treatment apparatus for generating a laser-induced refractive index change. Furthermore, the invention relates to a control device, which is configured to perform the method, to a computer program comprising commands, which cause the control device to execute the method, and to a computer-readable medium, on which such a computer program is stored.

### BACKGROUND

Treatment apparatuses and methods for controlling ophthalmological lasers for correcting an optical visual disorder and/or pathologically or unnaturally altered areas of the cornea are known in the prior art. Therein, pulsed lasers and a beam focusing device can, for example, be formed such that laser pulses effect a photodisruption and/or ablation in a focus area situated within the organic tissue to remove a tissue, in particular a tissue lenticule, from the cornea.

Besides the previously mentioned possibilities, the laser-induced refractive index change (LIRIC) is a further possibility of treating visual disorders. Herein, a tissue structure is changed by irradiation of laser pulses with short laser pulse length without generating an optical breakdown, whereby a refractive index of the concerned tissue structures changes and thus wavefronts can be adapted for correcting the visual disorder. In the laser-induced refractive index change, a separation of monomer compounds is in particular effected by pulses with laser pulse lengths between 10 fs and 500 fs, such that a collagen matrix is modified and densifies. Hereby, a refractive index increases, whereby structures can thus be generated in the cornea to shape an optical vision.

### SUMMARY

It is the object of the invention to provide further possibilities for adapting a visual disorder by a laser-induced refractive index change.

This object is solved by the independent claims. Advantageous embodiments of the invention are disclosed in the dependent claims, the following description as well as the figures.

The invention is based on the idea that longer-wave laser pulse lengths, which have an opposite effect on the refractive index, can also be used for the laser-induced refractive index change besides the use of short laser pulse lengths. Thus, a change of the structure of the cornea can be even better planned and performed.

An aspect of the invention relates to a method for providing control data for an ophthalmological laser of a treatment apparatus for generating a laser-induced refractive index change, wherein the method comprises the following steps performed by a control device. An appliance or an appliance component, in particular a processor or microprocessor, is to be understood by a control device, by which the following steps can be autonomously or partially autonomously performed: determining irradiation positions for irradiating by laser pulses for the laser-induced refractive index change is effected, wherein first and second laser pulse lengths are provided for irradiating the irradiation positions, wherein the first laser pulse length has a shorter laser pulse length than the second laser pulse length; setting desired irradiation positions to the second laser pulse length; and providing the control data for controlling the ophthalmological laser, which includes the second laser pulse length for the desired irradiation positions.

In other words, irradiation positions in an eye or a polymer material can be provided or preset to generate a structure by the laser-induced refractive index change, for example to compensate for a visual disorder. This means that the irradiation positions can include coordinates for an irradiation, at which the refractive index is to be changed. Furthermore, the ophthalmological laser can be formed to change a laser pulse length, wherein at least a first and a second laser pulse length are preset. Therein, the first laser pulse length, which may be in a laser pulse length range conventional for the laser-induced refractive index change, is shorter than the second laser pulse length, which may be in a laser pulse length range above the conventional laser pulse length range. Herein, the first laser pulse length can be predetermined such that a conventional refractive index change effect, which is to be performed by the laser-induced refractive index change, is achieved. This means that the first laser pulse length may be in a range between 10 fs and 500 fs. The second laser pulse length may be longer than the laser pulse length for the conventional refractive index change effect, for example beginning from 500 fs to 5 ps. In particular, it has been determined that a sign of the refractive index change is changed compared to the first laser pulse lengths, such that the refractive index can be reduced instead of increased at the second laser pulse length.

Accordingly, desired irradiation positions can be adjusted to the irradiation with the second laser pulse length such that further possibilities of changing the refractive index can be provided. In other words, at least a part of the originally planned irradiation positions cannot be irradiated by the conventional refractive index change effect by the first laser pulse length, but with the second laser pulse length.

Finally, control data for controlling the ophthalmological laser can be provided, which includes at least the irradiation positions, for which the second laser pulse length has been set. The ophthalmological laser can subsequently be controlled by the control data. The control data can include a respective dataset for positioning and/or for focusing individual laser pulses in the cornea. Additionally or alternatively, a respective dataset for adjusting at least one beam device for beam guidance and/or beam shaping and/or beam deflection and/or beam focusing of a laser beam of the respective laser can be included in the control data.

By the invention, the advantage arises that an accuracy and possibilities of adjusting for a desired refractive index change effect can be increased.

The invention also includes embodiments, by which additional advantages arise.

In an embodiment the irradiation positions are determined for an irradiation of a tissue of a human or animal eye. In other words, it can be provided that a cornea or an eye lens of a human or animal eye is, for example, to be irradiated.

In another embodiment the irradiation positions are determined for an irradiation of a polymer material, in particular of an artificial lens. In other words, it can be provided that a structure of an artificial material, in particular of a polymer material, is to be changed by the irradiation with the first and/or second laser pulse length, to for example produce an artificial lens. In particular, structures in the form of a Fresnel lens can be generated by the laser-induced refractive index change.

In another embodiment the second laser pulse length is set for all of the irradiation positions. In other words, it can be provided that instead of irradiating a part of the irradiation positions with the first laser pulse length and the other part with the second laser pulse length, all of the irradiation positions are set with the second laser pulse length. Thus, an inverse refractive index change effect can be provided for all of the irradiation positions. However, it can herein be further provided that a part or all of the irradiation positions are additionally irradiated with the first laser pulse length to provide a combination effect.

In another embodiment, the desired irradiation positions are set for exclusive irradiation with the second laser pulse length. This means that desired irradiation positions can only be irradiated with the second laser pulse length without the first laser pulse length being additionally provided for these preset irradiation positions.

In another embodiment the desired irradiation positions are set with a combination of the first and second laser pulse lengths. This means that the desired irradiation positions can be irradiated either sequentially or in mixed manner with the combination of the first and second laser pulse lengths. Herein, it is assumed that an intensification of the refractive index change effect can be achieved from the combination.

In another embodiment an opposite refractive index change is provided by the second laser pulse length compared to the first laser pulse length. This means that the second laser pulse length is predetermined such that a sign of the refractive index change effect arising thereby differs from a sign of the conventional refractive index change effect with the first laser pulse length. For example, if the first laser pulse length causes a positive refractive index change, then the second laser pulse length is provided in order to cause a negative refractive index change. Herein, it is assumed that the first laser pulse length causes a photochemical effect, which removes intracellular water in the tissue and thus changes a refractive index towards a refractive index of the tissue. With the longer second laser pulse length, it is assumed among other things that the irradiation results in heating of the tissue, which induces an additional absorption of water in the tissue, whereby the refractive index is changed towards the refractive index of water, wherein further effects can also be responsible for these refractive index changes.

In another embodiment the first laser pulse length is in a range between 10 fs and 500 fs and the second laser pulse length is in a range from 500 fs to 5 ps.

A further aspect relates to a method for controlling a treatment apparatus. Therein, the method includes the method steps of at least one embodiment of a method as it was previously described. Furthermore, the method for controlling the treatment apparatus also includes the step of transferring the provided control data to at least one ophthalmological laser of the treatment apparatus and controlling the treatment apparatus and/or the laser with the control data.

The respective method can include at least one additional step, which is executed if and only if an application case or an application situation occurs, which has not been explicitly described here. For example, the step can include the output of an error message and/or the output of a request for inputting a user feedback. Additionally or alternatively, it can be provided that a default setting and/or a predetermined initial state are adjusted.

A further aspect of the invention relates to a control device, which is formed to perform the steps of at least one embodiment of one or both of the previously described methods. Thereto, the control device can comprise a computing unit for electronic data processing such as, for example, a processor. The computing unit can include at least one microcontroller and/or at least one microprocessor. The computing unit can be configured as an integrated circuit and/or microchip. Furthermore, the control device can include an (electronic) data memory or a storage unit. A program code can be stored on the data memory, by which the steps of the respective embodiment of the respective method are encoded. The program code can include the control data for the respective laser. The program code can be executed by the computing unit, whereby the control device is caused to execute the respective embodiment. The control device can be formed as a control chip or control unit. The control device can, for example, be encompassed by a computer or computer cluster.

A further aspect of the invention relates to a computer program. The computer program includes commands, which, for example, form a program code. The program code can include at least one control dataset with the respective control data for the respective laser. Upon execution of the program code by a computer or a computer cluster, it is caused to execute the previously described method or at least one embodiment thereof.

A further aspect of the invention relates to a computer-readable medium (storage medium), on which the above-mentioned computer program and the commands thereof, respectively, are stored. For executing the computer program, a computer or a computer cluster can access the computer-readable medium and read out the content thereof. The storage medium is, for example, formed as a data memory, in particular at least partially as a volatile or a non-volatile data memory. A non-volatile data memory can be a flash memory and/or an SSD (solid state drive) and/or a hard disk. A volatile data memory can be a RAM (random access memory). For example, the commands can be present as a source code of a programming language and/or as assembler and/or as a binary code.

Further features and advantages of one of the described aspects of the invention can result from the embodiments of another one of the aspects of the invention. Thus, the features of the embodiments of the invention can be present in any combination with each other if they have not been explicitly described as mutually exclusive.

A further aspect relates to a treatment apparatus with at least one ophthalmological laser for a laser-induced refractive index change of a material, wherein the laser is configured to emit laser pulses with a first and second laser pulse length for the laser-induced refractive index change, wherein the first laser pulse length has a shorter laser pulse length than the second laser pulse length, wherein the treatment apparatus further comprises a control device which is configured to determining irradiation positions for irradiating the material for the laser-induced refractive index change, setting the second laser pulse length for predetermined irradiation positions, and controlling the laser to irradiate the predetermined irradiation positions with the second laser pulse length.

Consequently, the control device may set predetermined irradiation positions to irradiation with the second laser pulse length, so that further possibilities for changing the refractive index can be provided. In other words, at least some of the originally planned irradiation positions may be irradiated with the second laser pulse length rather than the conventional refractive index change effect using the first laser pulse length. The control device may comprise a computing unit for electronic data processing, such as a processor, to provide the irradiation positions and/or to control the ophthalmological laser. The computing unit may comprise at least one microcontroller and/or at least one microprocessor. The computing unit may be configured as an integrated circuit and/or microchip. Furthermore, the control device may comprise an (electronic) data memory or a memory unit. Program code, which encodes the control, may be stored in the data memory. The program code may, for example, include control data for the ophthalmological laser. The program code may be executed by the computing unit, causing the control device to execute the respective embodiment. The control device may be designed as a control chip or control unit. The control device may, for example, be comprised of a computer or computer network.

In an embodiment of the treatment apparatus, the ophthalmological laser comprises a pulse compression device that is configured to adjust the laser pulse length between the first and second laser pulse length. For example, a prism compressor, an optical grating compressor and/or dielectric mirrors, in particular Bragg mirrors or “chirped mirrors”, may be used as the pulse compressor device.

In another embodiment of the treatment apparatus, the ophthalmological laser comprises a dispersion element that is configured to change the laser pulse length between the first and second laser pulse length. The dispersion element may be controllable, which means that it may be moved in and out of a laser beam path of the ophthalmological laser in order to change between the first and second laser pulse length. The dispersion element may be, for example, a wedge that may be moved into the beam path, a crystal, an optical fiber and/or a deformable phase plate.

### BRIEF DESCRIPTION OF THE DRAWINGS

In the following, additional features and advantages of the invention are described in the form of advantageous execution examples based on the figure(s). The features or feature combinations of the execution examples described in the following can be present in any combination with each other and/or the features of the embodiments. This means, the features of the execution examples can supplement and/or replace the features of the embodiments and vice versa. Thus, embodiments are also to be regarded as encompassed and disclosed by the invention, which are not explicitly shown or explained in the figures, but arise from and can be generated by separated feature combinations from the execution examples and/or embodiments. Thus, embodiments are also to be regarded as disclosed, which do not comprise all of the features of an originally formulated claim or extend beyond or deviate from the feature combinations set forth in the relations of the claims. To the execution examples, there shows:

FIG. 1 depicts a treatment apparatus with an ophthalmological laser according to an exemplary embodiment.

FIG. 2 depicts a treatment apparatus with an ophthalmologic laser according to another exemplary embodiment.

FIG. 3 depicts a flow diagram for providing control data for the ophthalmological laser.

In the figures, identical or functionally identical elements are provided with the same reference characters.

### DETAILED DESCRIPTION

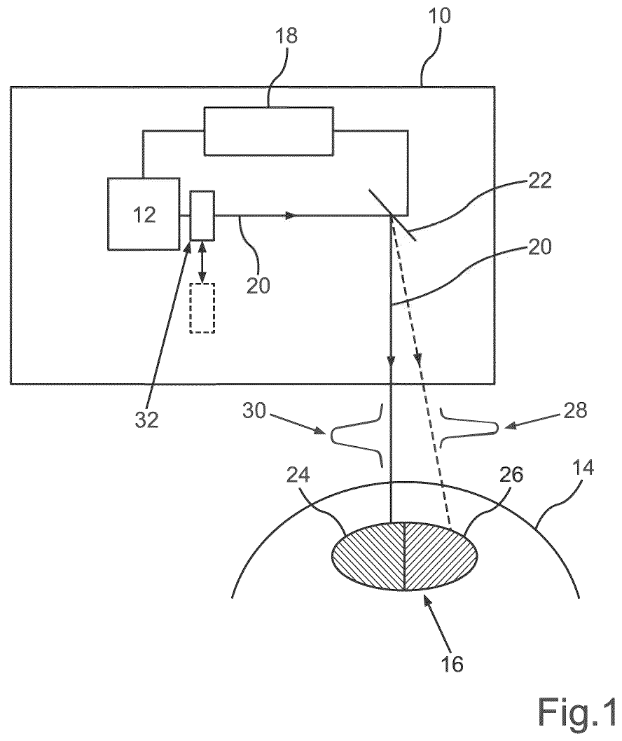

FIG. 1 shows a schematic representation of a treatment apparatus 10 with an ophthalmological laser 12 for generating a laser-induced refractive index change. In this example, the refractive index change can be planned in an eye 14, wherein the refractive index change can alternatively also be performed in a polymer material, for example in an artificial lens.

For the laser-induced refractive index change, irradiation positions 16 in the eye 14 can be provided by a control device 18 for generating a predetermined pattern, in particular in the form of control data, such that the laser 12 emits pulsed laser pulses in a pattern predefined by the irradiation positions 16 into the eye 14, to change the refractive index in the respective irradiation position. Herein, the control device 18 is illustrated as a part of the treatment apparatus 10, wherein an external control device can also be provided, for example as part of a planning device.

Furthermore, FIG. 1 shows that the laser beam 20 generated by the laser 12 can be deflected towards the eye 14 by a beam deflection device 22 such as, for example, a rotation scanner, to access the irradiation positions 16. The beam deflection device 22 can also be controlled by the control device 18.

The ophthalmological laser 12 may be a femtosecond laser, which is formed to emit laser pulses in a wavelength range between 300 nm and 1400 nm, for example between 700 nm and 1200 nm, at a respective pulse duration between 1 fs and 1 ns, for example between 10 fs and 10 ps, and a repetition frequency of greater than 10 kHz, for example between 100 kHz and 100 MHz. In particular, the ophthalmological laser 12 can at least be formed to radiate a first pulse length 28, which is in a range between 10 fs and 500 fs, and a second laser pulse length 30, which is in a range from 500 fs to 5 ps. In addition, the control device 18 optionally comprises a storage device (not illustrated) for at least temporary storage of at least one control dataset, wherein the control dataset or datasets include(s) control data for positioning and/or for focusing individual laser pulses in the eye 14.

In the irradiation of the irradiation positions 16 for the laser-induced refractive index change, an effect of short laser pulse lengths, which can in particular be provided with the first laser pulse length 28, was exclusively exploited up to now. By the first laser pulse length 28, an increase of the refractive index can in particular be achieved, whereby preset patterns can be generated in the eye 14, in particular the cornea of the eye 14, which can compensate for visual disorders, such as, for example, higher order aberrations. By temporally longer laser pulses, as they can be provided by the second laser pulse length 30, a further effect can be achieved, in which the refractive index reduces. Therefore, it can be provided that desired irradiation positions 24 of the original irradiation positions 16 are planned for irradiating with the second laser pulse length 30, in particular depending on a pattern to be achieved, to increase the possibilities in generating a pattern for the laser-induced refractive index change. In some embodiments, the desired irradiation positions 24 of the original irradiation positions 16 can be determined in real time, predetermined or preset.

Further irradiation positions 26, which are not planned for irradiating with the second laser pulse length 30, can then, for example, be irradiated with the first laser pulse length 28. Alternatively, all of the originally planned irradiation positions 16 can also be irradiated with the second laser pulse length 30, to achieve the effect of the opposite refractive index change by long laser pulses for the entire irradiation pattern, or all or a part of the irradiation positions 16 can be irradiated by a combination of the first and second laser pulse lengths 28, 30, to achieve a combination effect.

In order to change the laser pulse lengths between the first and second laser pulse lengths, the treatment apparatus 10, in particular the ophthalmologic laser 12, may comprise a dispersion element 32 which may be moved in and out of the beam path of the laser beam 20. The moving of the dispersion element 32 may, for example, be controlled by an electric motor. The dispersion element 32 may be an element that is transparent to the laser radiation, for example a glass, a crystal or a plastic. For example, a retractable wedge or an optical fiber may be introduced into the beam path as a dispersion element 32.

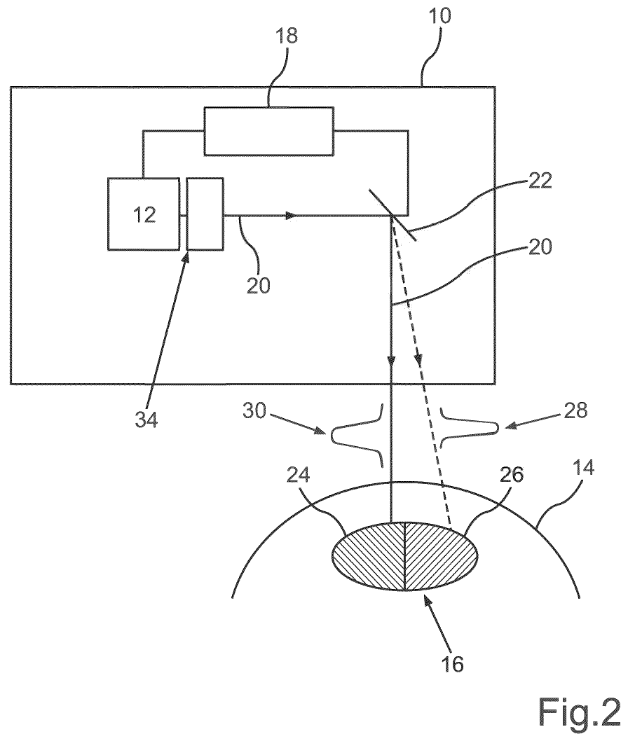

FIG. 2 shows a similar treatment apparatus 10 as in FIG. 1, wherein in this embodiment, a pulse compressor device 34 is provided in the beam path of the laser beam 20 for changing the laser pulse length between the first and second laser pulse length. The pulse compressor device 34 may comprise an arrangement of gratings, prisms or dielectric mirrors by which the pulse length is adjusted. For example, a prism compressor or a grid compressor may thus be provided. Alternatively or additionally, at least one deformable phase plate may be provided in the beam path. With the deformable phase, a path length of the laser beam 20, in particular spatial components of the laser beam 20, may be adjusted by electrical control through a dispersive medium, whereby the different laser pulse lengths may be provided.

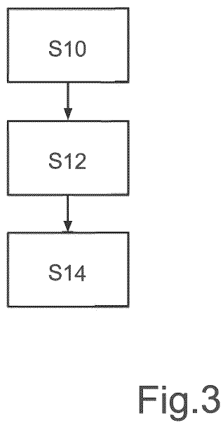

FIG. 3 shows a schematic method diagram for providing control data for the ophthalmological laser 12 of the treatment apparatus 10 for generating a laser-induced refractive index change.

In a step S10, irradiation positions 16 for irradiating by laser pulses for the laser-induced refractive index change can be determined. The irradiation positions can, for example, be ascertained from a preset pattern to be generated, wherein the irradiation positions comprise a location or coordinates for placement of the laser pulses. In other words, the control device 18 can convert a planned pattern to coordinates for controlling by the laser 12 and/or the beam deflection device 22.

In a step S12, it can be set for each irradiation position 16, with which laser pulse length it is to be irradiated. In particular, desired irradiation positions 24 can be present, which are to be irradiated with the second laser pulse length 30, and further irradiation positions 26, which are to be irradiated with the first laser pulse length 28. Herein, it can be provided that an opposite refractive index change is provided by the second laser pulse length 30 compared to the first laser pulse length 28. The desired irradiation positions 24, which may be preset for the irradiation with the second laser pulse length 30, can, for example, be ascertained from a planned pattern and be adapted to a phase of wavefronts to be corrected. Besides the previously described strict division to the laser pulse lengths for desired irradiation positions 24, a combination of the first and second laser pulse lengths 28, may also be preset for desired irradiation positions 24 or all irradiation positions 16, to obtain intensified refractive index change effects.

After the laser pulse lengths have been set for the respective irradiation positions, the control data for controlling the ophthalmological laser 12 can be provided in a step S14. This means that the control data can be transferred to the laser 12 by the control device 18 for controlling, which can then emit the laser pulses for the corresponding irradiation positions 16 with the set laser pulse lengths.

Overall, the examples show, how an improved generation of a laser-induced refractive index change can be provided by the invention.

1. A method for providing control data for an ophthalmological laser of a treatment apparatus for generating a laser-induced refractive index change, wherein the method comprises the following steps performed by a control device:
determining irradiation positions for irradiation by laser pulses for the laser-induced refractive index change, wherein a first laser pulse length and a second laser pulse length are provided for the irradiation of the irradiation positions, wherein the first laser pulse length has a shorter laser pulse length than the second laser pulse length;
setting desired irradiation positions to the second laser pulse length;
providing the control data for controlling the ophthalmological laser, which includes the second laser pulse length for the desired irradiation positions.

2. The method according to claim 1, wherein the irradiation positions are determined for irradiation of a tissue of a human or animal eye.

3. The method according to claim 1, wherein the irradiation positions are determined for irradiation of a polymer material, in particular of an artificial lens.

4. The method according to claim 1, wherein all of the irradiation positions are set to the second laser pulse length.

5. The method according to claim 1, wherein the desired irradiation positions are set for exclusive irradiation with the second laser pulse length.

6. The method according to claim 1, wherein the desired irradiation positions are set for a combination of the first and second laser pulse lengths.

7. The method according to claim 1, wherein the second laser pulse length is configured to effect an opposite sign refractive index change compared to the first laser pulse length.

8. The method according to claim 1, wherein the first laser pulse length is in a range between 10 fs and 500 fs and the second laser pulse length is in a range from 500 fs to 5 ps.

9. A control device, which is configured to perform a method according to claim 1.

10. (canceled)

11. A computer-readable medium for storing a computer program, the computer program comprising commands which cause a control device to execute the method according to claim 1.

12. A treatment apparatus with at least one ophthalmological laser for a laser-induced refractive index change of a material, wherein the at least one ophthalmological laser is configured to emit laser pulses with a first laser pulse length and a second laser pulse length for the laser-induced refractive index change, wherein the first laser pulse length has a shorter laser pulse length than the second laser pulse length, wherein the treatment apparatus further comprises a control device which is configured to determining irradiation positions for irradiating the material for the laser-induced refractive index change, setting the second laser pulse length for desired irradiation positions, and controlling the ophthalmological laser to irradiate the desired irradiation positions with the second laser pulse length.

13. The treatment apparatus according to claim 12, wherein the at least one ophthalmological laser comprises a pulse compression device that is configured to adjust the laser pulse length between the first laser pulse length and the second laser pulse length.

14. The treatment apparatus according to claim 12, wherein the at least one ophthalmological laser comprises a dispersion element that is configured to change the laser pulse length between the first laser pulse length and the second laser pulse length.
